(19) United States
(12) Patent Application Publication
(10) Pub. No.: US 2010/0036333 A1
(43) Pub. Date: Feb. 11, 2010

(54) FLUID LEVEL SENSOR FOR A CONTAINER OF A NEGATIVE PRESSURE WOUND TREATMENT SYSTEM

(76) Inventors: Albert A. Schenk, III, Trevor, WI (US); Patrick E. Eddy, Allendale, MI (US); David K. Platt, Mt. Prospect, IL (US); Joseph A. Carmichael, Burlington, WI (US); Terry S. Callaghan, Newaygo, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/262,474

(22) Filed: Oct. 31, 2008

Related U.S. Application Data
(63) Continuation-in-part of application No. 12/187,114, filed on Aug. 6, 2008.

(51) Int. Cl. A61M 1/00

(52) U.S. Cl. 604/313; 604/318

(57) ABSTRACT

An apparatus is provided for collecting medical waste fluids. The apparatus includes: a medical waste collection container for collecting medical waste fluids; first and second electrically conductive sensing elements disposed inside the container so as to be exposed to fluid collected in the container, where the first and second sensing elements are located inside the container at a position corresponding to a full fluid level at which point the container is full of fluid; and an electronic circuit coupled to the sensing elements for generating a full alarm signal when the electronic circuit determines that current can flow between the first and second sensing elements as a result of fluid in the container reaching the full fluid level and thereby completing a current path between the first and second sensing elements.

### CROSS-REFERENCE TO RELATED APPLICATION

This application is a continuation-in-part of U.S. patent application Ser. No. 12/187,114, filed on Aug. 6, 2008, the entire disclosure of which is incorporated herein by reference.

### BACKGROUND OF THE INVENTION

The present invention is generally directed to a fluid level sensor system for a container and, for some embodiments, to a fluid level sensor system for a container used in a negative pressure wound treatment system.

Wound treatment systems that treat a wound using a vacuum are known. Examples of such systems are disclosed in U.S. Pat. Nos. 4,382,441, 4,392,858, 4,655,754, 4,826,494, 4,969,880, 5,100,396, 5,261,893, 5,527,293, 5,636,643, 5,645,081, 6,071,267, 6,117,111, 6,135,116, 6,142,982, 6,174,306, 6,345,623, 6,398,767, 6,520,982, 6,553,998, 6,814,079, 7,198,046, and 7,216,651, the entire disclosures of which are incorporated herein by reference. These systems utilize either a manual pump or a portable vacuum pump to draw air and fluid from the wound site. The fluid drawn is typically collected in a container. Because such containers will overflow if not monitored closely, many of the pumps available today utilize a mechanical shut off mechanism, such as a float coupled to a valve. A problem with these types of mechanisms is that they may shut off the system and thus stop the negative pressure wound treatment until such time that a nurse or other caretaker removes and empties or replaces the container. This interruption in application of the treatment is undesirable.

### SUMMARY OF THE INVENTION

According to one aspect of the present invention, an apparatus is provided for collecting medical waste fluids. The apparatus comprising: a medical waste collection container for collecting medical waste fluids; first and second electrically conductive sensing elements disposed inside the container so as to be exposed to fluid collected in the container, where the first and second sensing elements are located inside the container at a position corresponding to a full fluid level at which point the container is full of fluid; and an electronic circuit coupled to the sensing elements for generating a full alarm signal when the electronic circuit determines that current can flow between the first and second sensing elements as a result of fluid in the container reaching the full fluid level and thereby completing a current path between the first and second sensing elements.

According to another aspect of the invention, a system is provided for the treatment of wounds by applying a negative pressure to a wound site. The system comprising: an electronically controlled suction regulator; a valve connected to the suction regulator for supplying a suction at an output; a medical waste collection container for collecting medical waste fluids and coupled to the output of the valve; first and second electrically conductive sensing elements disposed inside the container so as to be exposed to fluid collected in the container, where the first and second sensing elements are located inside the container at a position corresponding to a full fluid level at which point the container is full of fluid; and a control circuit coupled to the valve and the first and second sensing elements for generating control signals for controlling the valve and for generating a full alarm signal when the control circuit determines that current can flow between the first and second sensing elements as a result of fluid in the container reaching the full fluid level and thereby completing a current path between the first and second sensing elements, wherein the control circuit further generates a control signal to interrupt the supply of the suction at the output when a full alarm signal is generated.

According to another aspect of the invention, a method is provided for detecting whether a container is full of medical waste fluids comprising the steps of: providing first and second electrically conductive sensing elements inside the container so as to be exposed to fluid collected in the container, where the first and second sensing elements are located inside the container at a position corresponding to a full fluid level at which point the container is full of fluid; and generating a full alarm signal when electrical current can flow between the first and second sensing elements as a result of fluid in the container reaching the full fluid level and thereby completing a current path between the first and second sensing elements.

These and other aspects, features, advantages, and objects of the present invention will be further understood and appreciated by those skilled in the art by reference to the following specification, claims, and appended drawings.

### BRIEF DESCRIPTION OF THE DRAWINGS

In the drawings:

FIG. 1 is a side view of an apparatus constructed according to a first embodiment;

FIG. 2 is a side view of an apparatus constructed according to a second embodiment;

FIG. 3 is an electrical circuit diagram in block and schematic form of a fluid level sensing system used in the first and second embodiments;

FIG. 4 is a side view of an apparatus constructed according to a third embodiment;

FIG. 5 is an electrical circuit diagram in block and schematic form of a fluid level sensing system used in the third embodiment;

FIG. 6A is a perspective view of a portion of a wound treatment system according to the present invention;

FIG. 6B is a front view of a portion of a wound treatment system according to the present invention;

FIG. 7 is a fluid flow and electrical circuit diagram in block form of a wound treatment system according to the present invention;

FIG. 8 is an electrical circuit diagram in block form of a suction regulator according to the present invention;

FIG. 9 is a perspective view of a wound dressing portion that may be used in the inventive wound treatment system;

FIG. 10 is a cross-sectional view of the wound dressing portion shown in FIG. 9 taken along line X-X;

FIG. 11 is a top view of the wound dressing portion shown in FIG. 9; and

FIG. 12 is a cut-away perspective view of a portion of the bottom surface of a drape of the wound dressing portion shown in FIGS. 9 and 11.

### DETAILED DESCRIPTION OF THE PREFERRED EMBODIMENTS

Reference will now be made in detail to the presently preferred embodiments of the invention, examples of which are illustrated in the accompanying drawings. Wherever possible, the same reference numerals will be used throughout the drawings to refer to the same or like parts.

For purposes of description herein, the terms “upper,” “lower,” “right,” “left,” “rear,” “front,” “vertical,” “horizontal,” “top,” “bottom,” and derivatives thereof shall relate to the invention as shown in the drawings. However, it is to be understood that the invention may assume various alternative orientations, except where expressly specified to the contrary. It is also to be understood that the specific devices illustrated in the attached drawings and described in the following specification are simply exemplary embodiments of the inventive concepts defined in the appended claims. Hence, specific dimensions, proportions, and other physical characteristics relating to the embodiment disclosed herein are not to be considered as limiting, unless the claims expressly state otherwise.

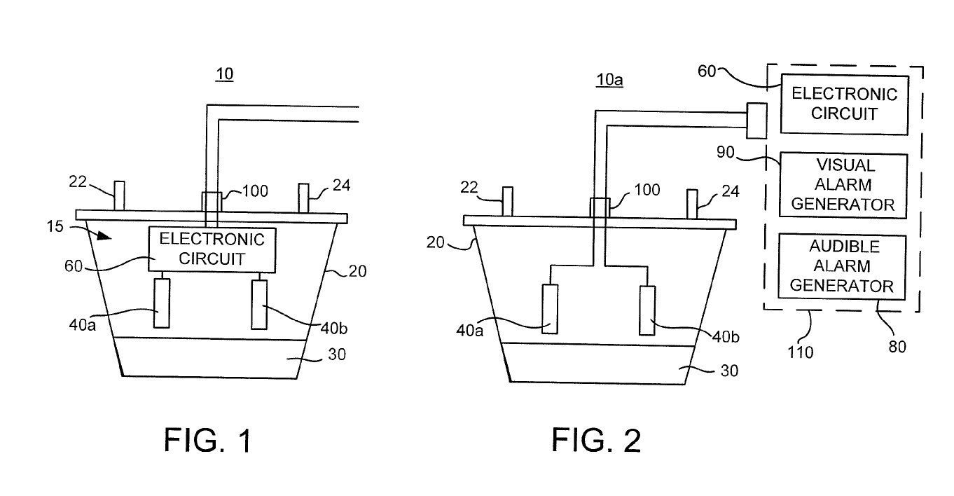

According to a first embodiment of the present invention shown in FIG. 1, an apparatus 10 for collecting medical waste may include a medical waste collection container 20 for collecting medical waste fluids 30 and a fluid level sensing system 15, which may include a first electrically conductive sensing element 40a and second electrically conductive sensing element 40b both disposed inside container 20 so as to be exposed to fluid collected in container 20. First and second sensing elements 40a and 40b are located inside container 20 at a position corresponding to a full fluid level at which point container 20 is full of fluid. Fluid level sensing system 15 may further include an electronic circuit 60 coupled to sensing elements 40a and 40b for generating a full alarm signal when electronic circuit 60 determines that current can flow between first and second sensing elements 40a and 40b as a result of fluid in container 20 reaching the full fluid level and thereby completing a current path between first and second sensing elements 40a and 40b. Thus, when a continuous pool of liquid 30 makes contact with both sensing elements 40a and 40b, an electronic signal is relayed to electronic circuit 60, which will, in turn, initiate an alarm condition.

Container 20 may include a liquid inlet 22 through which medical waste fluid is received, and a vent 24, which may be connected to suction equipment as described further below.

According to the first embodiment, electronic circuit 60 may be disposed inside container 20 so as to be completely self-contained and mounted within any standard or custom medical waste collection container at a factory preset level. However, according to a second embodiment shown in FIG. 2, electronic circuit 60 may be mounted external to container 20 and possibly even remote from container 20.

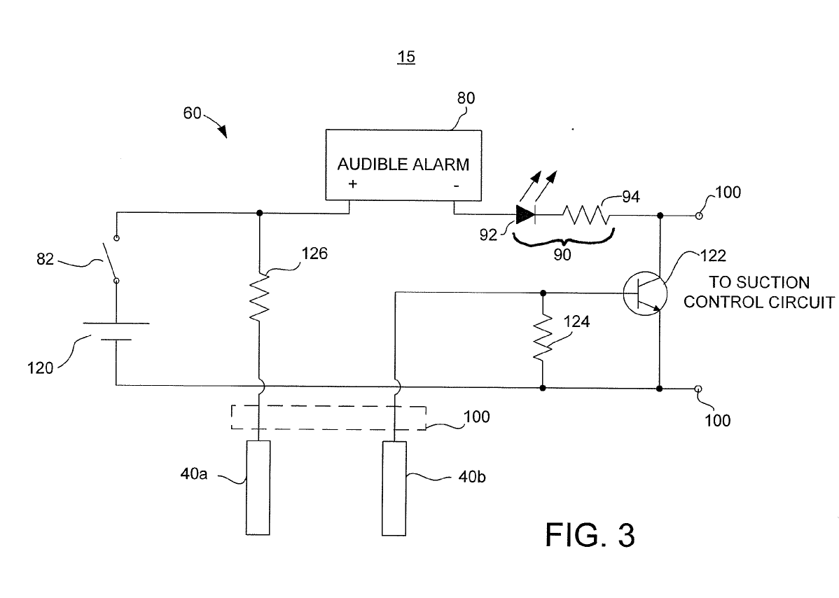

As shown in schematic form in FIG. 3, the apparatus may further include an audible alarm 80 that may be an integral component of electronic circuit 60 or may be remotely located from electronic circuit 60. Audible alarm 80 may be configured to respond to the full alarm signal by producing an audible alarm sound. Audible alarm 80 may include a piezo electric noise emitter. Apparatus 10 may further include a switch 82 for silencing audible alarm 80 when actuated or for preparing the container for disposal. Audible alarm 80 may also be mounted remotely from container 20.

As also shown in FIGS. 2 and 3, apparatus 10a may further include a visual alarm generator 90 that may be an integral component of electronic circuit 60 or may be remotely located from electronic circuit 60. Visual alarm generator 90 may be configured to respond to the full alarm signal by producing a visible alarm. Visual alarm generator 90 may include an LCD visual indicator and/or an LED visual indicator. Visual alarm generator 90 may be mounted remotely from container 20 as shown in FIG. 2.

Apparatus 10 may thus include a connector 100 for connecting the components within container 20 to those outside of container 20. For example, according to the first embodiment wherein electronic circuit 60 is disposed within container 20, connector 100 may be provided to connect to any external audio/visual alarms or to any other device (as described further below) that may be responsive to a full container alarm signal. According to the second embodiment, connector 100 may be provided to connect sensing elements 40a and 40b to an externally mounted electronic circuit 60. As shown in FIG. 2, electronic circuit 60, audible alarm generator 80, and visual alarm generator 90 may be disposed in a housing 110 separate from container 20. Housing 110 may be configured for attachment to container 20 or for remote mounting from container 20. Electronic circuit 60 and alarms 80 and 90 may be incorporated in a stand-alone housing (i.e., 110) or integrated with other medical equipment.

Electronic circuit 60 may further include a battery 120 for providing power for the circuit. Battery 120 may be coupled in series with switch 82, audible alarm 80, and visual alarm generator 90 such that switch 82 may interrupt the supply of power to alarms 80 and 90. Battery 120 would be desirable, particularly when electronic circuit 60 is mounted within container 20. However, power may be supplied from outside the container by means of connector 100. Such external power may be supplied from a battery, from an AC-DC converter coupled to an AC power outlet, or from a power supply of some other equipment that may be associated with apparatus 10. Apparatus 10 may be configured as shown and described below in order to remain dormant with no power demand from battery 120 until a continuous pool of liquid contacts both sensing elements 40a and 40b.

With further reference to FIG. 3, electronic circuit 60 may further include a switching transistor 122 (preferably an NPN transistor) coupled in series with battery 120, switch 82, optional audible alarm 80, and optional visual alarm 90. Transistor 122 thus may have its collector coupled to the positive terminal of battery 120 (optionally via audible alarm 80 and visual alarm 90), its drain coupled to the negative terminal of battery 120, and its base coupled to second sensing element 40b and to the negative terminal of battery 120 via a resistor 124 (which may have a resistance of 250 kΩ). First sensing element 40a is coupled to the positive terminal of battery 120 via a resistor 126 (which may have a resistance of 5 kΩ). Transistor 122 is non-conducting when fluid 30 does not provide a current path between first and second sensing elements 40a and 40b. However, when fluid 30 reaches a level that it does provide a current path between first and second sensing elements 40a and 40b, the voltage at the base of transistor 122 is pulled high causing it to conduct current and thereby complete the series connection of battery 120 and any optional alarms 80/90.

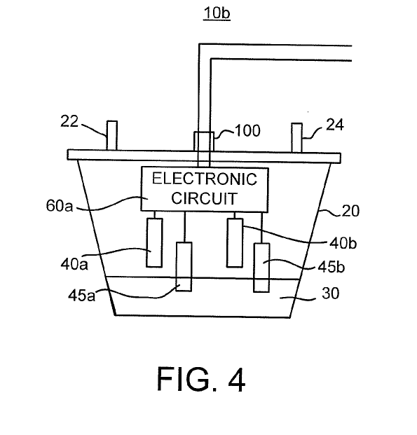

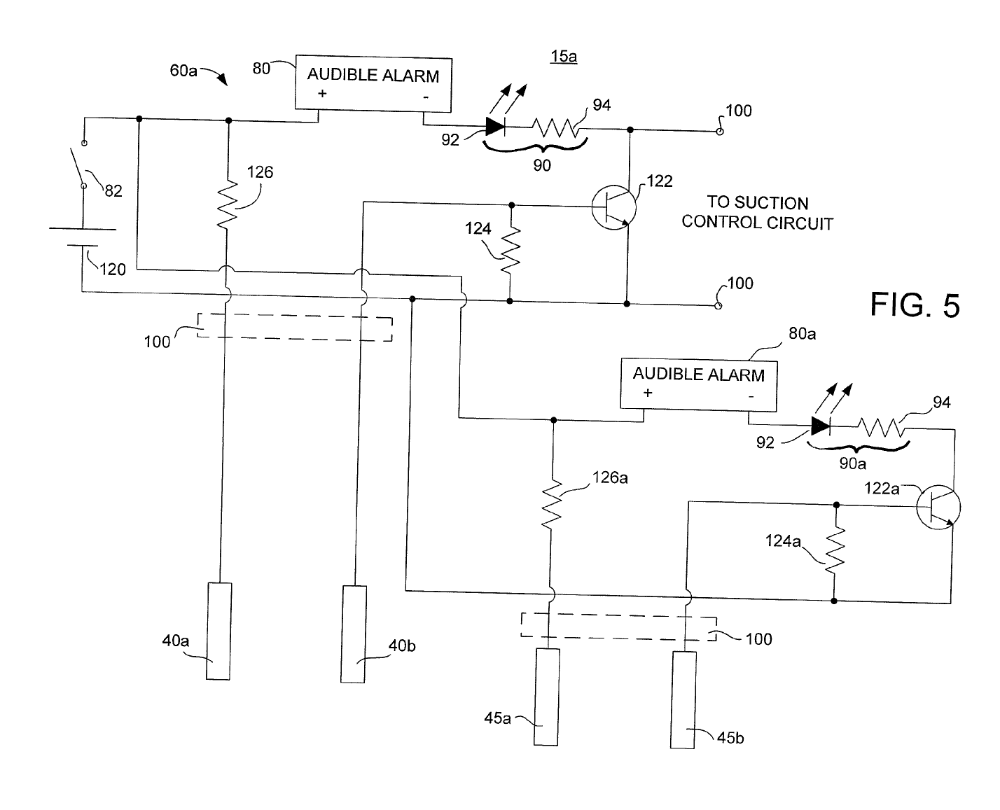

FIGS. 4 and 5 show another embodiment of apparatus 10b in which third and fourth electrically conductive sensing elements 45a and 45b, respectively, are disposed inside container 20 so as to be exposed to fluid 30 collected in container 20. Third and fourth sensing elements 45a and 45b are located inside container 20 at a position corresponding to a nearly full fluid level at which point container 20 is nearly full of fluid 30, but not yet full of fluid. Electronic circuit 60a is coupled to third and fourth sensing elements 45a and 45b for generating an initial alarm signal when electronic circuit 60a determines that current can flow between third and fourth sensing elements 45a and 45b as a result of fluid 30 in container 20 reaching the nearly full fluid level and thereby completing a current path between third and fourth sensors 45a and 45b.

As shown in FIG. 5, an audible alarm 80a may be provided to produce an audible alarm sound in response to the initial alarm signal. Audible alarm 80a may be separate from audible alarm 80 or may be the same alarm component that responds to both the initial and full alarm signals. As shown in FIGS. 3 and 5, visual alarm generators 90 and 90a may include an LED 92 in series with a resistor 94, which may have a resistance of 240Ω.

As also shown in FIG. 5, a visual alarm generator 90a may be provided to produce a visible alarm in response to the initial alarm signal. Visual alarm generator 90a may be separate from visual alarm generator 90 or may be the same alarm component that responds to both the initial and full alarm signals.

Sensing elements 40a, 40b, 45a, and 45b may be made of any electrically conductive material, preferably a material that does not corrode in the presence of the medical waste fluid. A preferred material being copper.

As shown in FIG. 5, electronic circuit 60a may further include a switching transistor 122a coupled in series with battery 120, switch 82, optional audible alarm 80a, and optional visual alarm 90a. Transistor 122a thus may have its collector coupled to the positive terminal of battery 120 (optionally via audible alarm 80a and visual alarm 90a), its drain coupled to the negative terminal of battery 120, and its base coupled to fourth sensing element 45b and to the negative terminal of battery 120 via a resistor 124a. Third sensing element 40a is coupled to the positive terminal of battery 120 via a resistor 126a. Transistor 122a is non-conducting when fluid 30 does not provide a current path between third and fourth sensing elements 45a and 45b. However, when fluid 30 reaches a level that it does provide a current path between third and fourth sensing elements 45a and 45b, the voltage at the base of transistor 122a is pulled high causing it to conduct current and thereby complete the series connection of battery 120 and any optional alarms 80a/90a.

As described further below, apparatus 10, 10a, 10b or associated equipment may be equipped with a valve for interrupting a flow of medical waste fluid into container 20 in response to the full alarm signal. If the third and fourth sensing elements 45a and 45b are provided, the valve would not be responsive to the initial alarm signal, but only the full alarm signal. In this way, apparatus 10 may first warn that container 20 is nearly full using an audible and/or visual alarm prior to closing the valve to stop the flow of waste fluid into container 20 in response to a full alarm signal. Audible and/or visual alarms may also be generated in response to the full alarm signal so as to indicate that the container should be emptied or replaced.

As described further below, apparatus 10, 10a, 10b or associated equipment may be equipped with a vacuum pump for drawing medical waste fluid into container 20. Such a vacuum pump may be coupled to vent 24 so as to draw fluid 30 into container via inlet 22. The flow of fluid 30 into container 20 may thus be halted by turning off or venting the vacuum pump in response to the full alarm signal.

A system is disclosed below for the treatment of wounds that utilizes apparatus 10. In general, the wound treatment system includes: a wound dressing that is directly in contact with the wound being treated; a wound drape that extends over the wound dressing and creates a seal around the wound and the wound dressing; a vacuum source for generating a vacuum; vacuum conduit for connecting the vacuum source to the interior of the wound drape; and a containment apparatus 10 that will contain solids and liquids passing through the vacuum conduit, but let gaseous materials pass to the atmosphere.

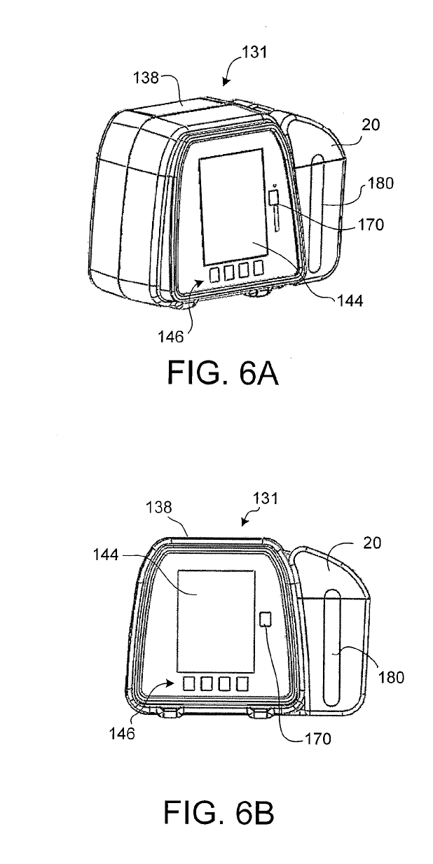

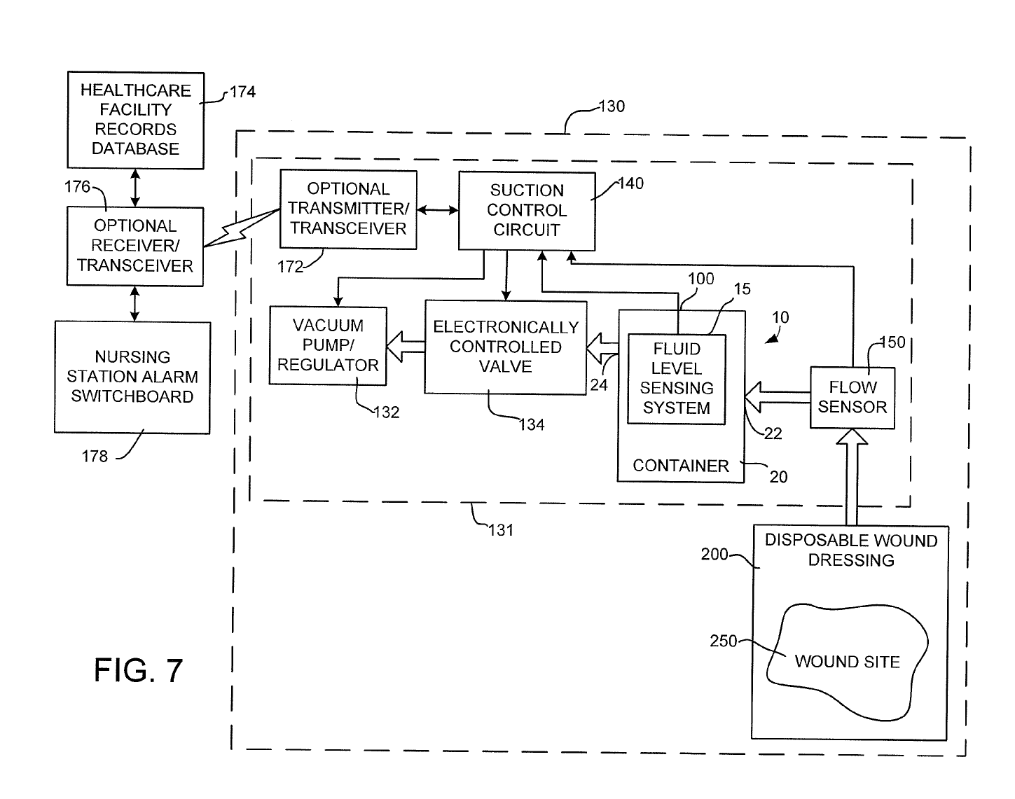

As shown in FIGS. 6A, 6B, and 7, a system 130 is provided for treatment of wounds applying a negative pressure to a wound site that may include: an electronically controlled suction regulator/pump 131 comprising a vacuum regulator/pump 132; a valve 134 (such as an electronically controlled three-way solenoid valve or a pneumatic valve) connected to vacuum regulator/pump 132 for supplying a suction at an output; a medical waste collection container 20 for collecting medical waste fluids from a patient's wound and coupled to the output of valve 134; first and second electrically conductive sensing elements 40a and 40b, respectively, disposed inside container 20 so as to be exposed to fluid 30 collected in container 20, where first and second sensing elements 40a and 40b are located inside container 20 at a position corresponding to a full fluid level at which point container 20 is full of fluid 30; and a control circuit 136 coupled to valve 134 and first and second sensing elements 40a and 40b for generating control signals for controlling valve 134 and for generating a full alarm signal when control circuit 136 determines that current can flow between first and second sensing elements 40a and 40b as a result of fluid 30 in container 20 reaching the full fluid level and thereby completing a current path between first and second sensing elements 40a and 40b, wherein control circuit 136 further generates a control signal to interrupt the supply of the suction at the output when a full alarm signal is generated.

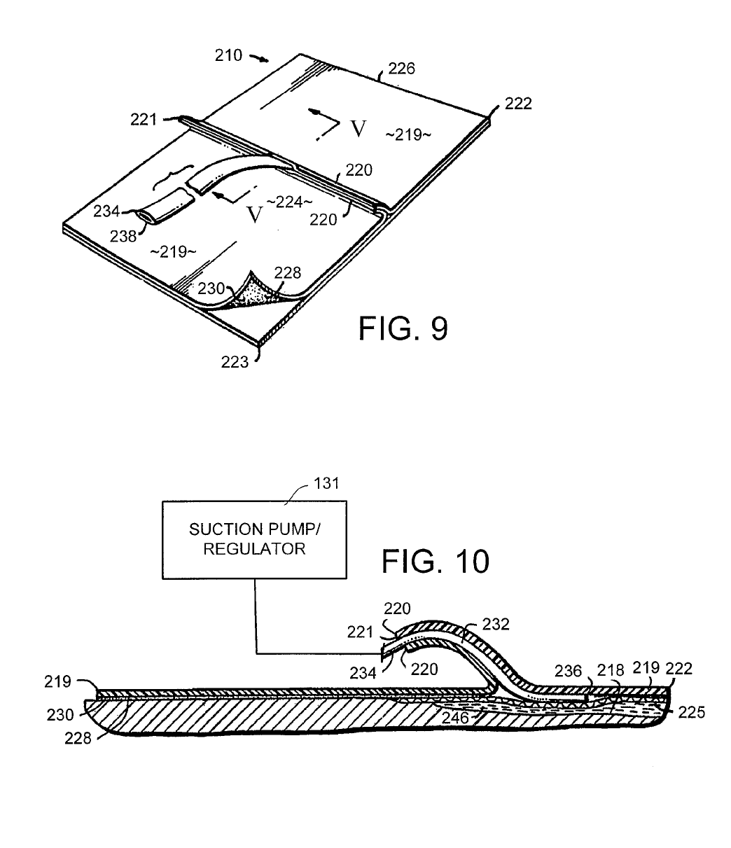

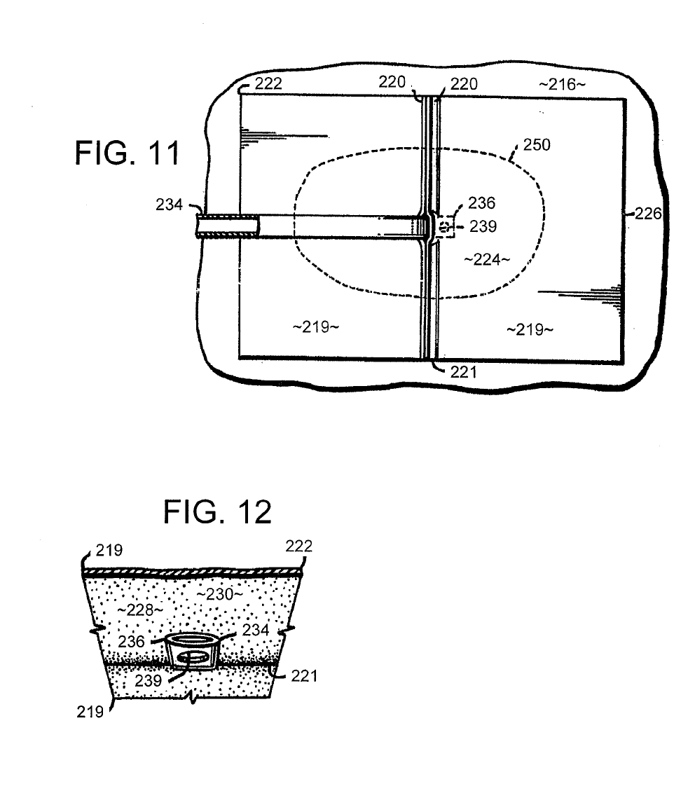

As shown in FIG. 7, system 130 may further include a disposable wound dressing 200 for application to a wound site 250 of a patient. Wound dressing 200 may be coupled to valve 134. As discussed further below with respect to FIGS. 9-13, dressing 200 effectively seals the wound site so that a negative pressure may be maintained at the wound site 250. Wound dressing 200 may comprise a wound dressing pad 225 for placing over the wound; and a wound drape 222 provided over wound dressing pad 225 and wound site 250 for securing wound dressing pad 225 and sealing the wound site 250 for application of the negative pressure.

Electronically controlled suction regulator/pump 131 may further comprise a flow sensor 150, such as a pressure transducer, connected to suction control circuit 140 for monitoring the negative pressure applied to a patient or device and an optional transmitter or transceiver 172 for transmitting information to a healthcare facility database 174 via an optional receiver or transmitter 176 or to a nursing station alarm switchboard 178 via transmitter 176.

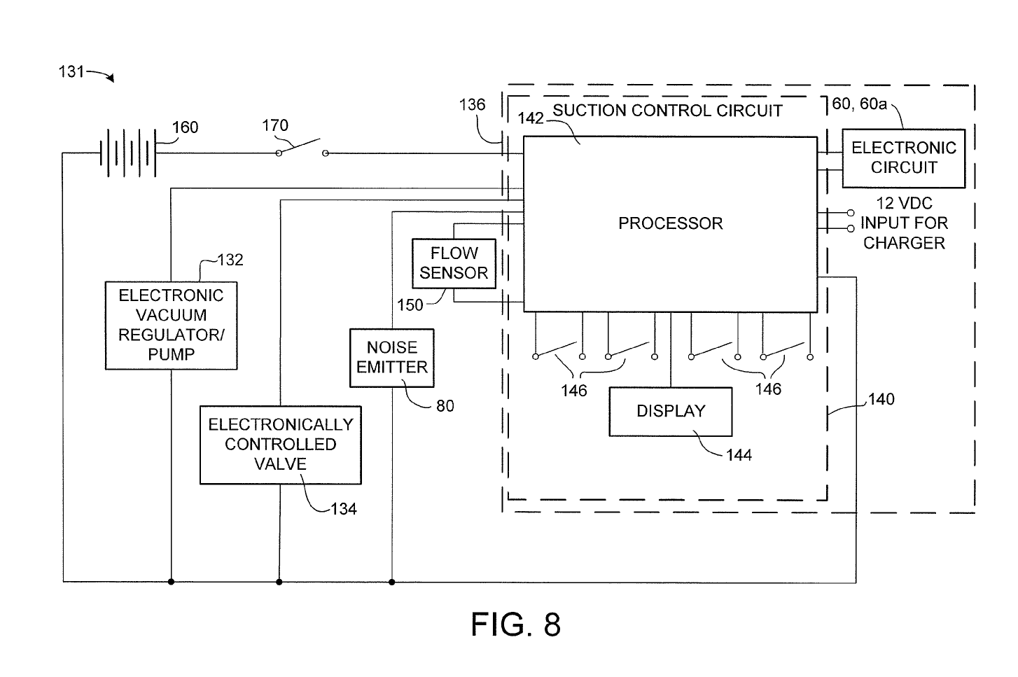

As shown in FIG. 8, control circuit 136 may include an electronic circuit 60 or 60a, which is coupled to first and second sensing elements 40a and 40b, and a suction control circuit 140 coupled to electronic circuit 60 or 60a and to valve 134 and vacuum regulator/pump 132. Suction control circuit 140 is powered by electrical current for controlling various components of the regulator and for generating control signals for controlling valve 134 so that the source of vacuum supplied to a patient or device for predetermined periods of time is able to deliver constant or intermittent vacuum.

As shown in FIG. 8, suction control circuit 140 may comprise a programmable digital processor 142 and a liquid crystal display or similar technology display panel 144 connected to the other electronic circuitry. Suction control circuit 140 may further include an end user interface 146 such as a touch pad with one or more switches, connected to processor 142. Processor 142 may be programmable to turn the electronics on and off at prescribed times. In addition, end user interface 146 may be configured to allow an end user to select various settings that may be employed to adjust the characteristics (i.e., timing cycle, intermittent mode, continuous mode, pressure, etc.) of the suction produced at the output of suction regulator/pump 131. In addition, the electronically controlled suction regulator/pump may provide the ability to lock out negative pressure settings so that the patients cannot change settings by the healthcare providers.

Suction regulator/pump 131 may further comprise a rechargeable battery 160 and a main power switch coupled in series with suction control circuit 140 so as to selectively power the portable device. Suction regulator/pump 131 may also include a pair of terminals for connection to a 12 VDC input for charging the battery. Suction control circuit 140 may include an AC to DC converter and regulating circuitry that may be connected to these terminals such that regulated DC power is supplied to the electronic circuitry and the battery 160.

Suction regulator/pump 131 may be constructed as a single unit having a housing 138 with integrated regulation of the vacuum and a container 20 that will contain solids and liquids, but let gaseous materials pass to the atmosphere. The purpose is to provide a safe method of providing either constant or intermittent/modulated vacuum to a physician or health provider for use on a patient or connection to a device that may or may not be used on a patient. This device is electronically controlled and will perform various functions including the ability to lock-out users from changing settings to alarming functions for safety and efficacy.

Container 20 includes the fluid level sensing system 15 including electronic circuit 60 described above, which may be connected to processor 142 for generating an audible alarm using a noise emitter 80 and/or a visual alarm 90 using an LED or LCD 144 to indicate that the contents have reached a particular level. Container 20 may be used with a ‘gel pack’ and or a porous filter.

Housing 138 and container 20 may be made from polymers for light weight and impact resistance. Further, container 20 may be replaceable and thus disposable and may contain about 250-1500 ml. Container 20 may be removable and may be sealed with a gasket, o-ring, or similar sealing apparatus. Container 20 may be frosted to obstruct portions of view but is clear in specific areas 180 to view contents and compare to a scale such as but not limited to, milliliters. More specifically, container 20 may be made of a plastic that is frosted except for the fluid level viewing window 180, which is clear. Container 20 may be a portion less than a ¾ circle but more than a ¼ circle and may be keyed to fit housing 138 with an integral incorporated into basis conduit/hose with a press fit cradle.

Electronically controlled suction regulator/pump 131 may thus comprise a safe regulation system with integrated container 20 and device for preventing liquids from leaving the container 20 thus containing possible contaminates. Further, the electronically controlled suction regulator may comprise integrated electronics that will regulate between 0 and 600 mmHg and provide the ability to modulate/intermittent between negative pressure and atmospheric pressure.

Electronically controlled suction regulator/pump 131 includes component 132 that may be an internal vacuum pump or a vacuum regulator that connects to an external vacuum source such as the built-in central vacuum system of a healthcare facility, central vacuum pump remotely located from the suction regulator, or a separate portable vacuum pump. A system that includes such a vacuum regulator is disclosed in Commonly assigned U.S. patent application Ser. No. 12/187,114, entitled “WOUND TREATMENT SYSTEM AND SUCTION REGULATOR FOR USE THEREWITH” and filed on Aug. 6, 2008 by Patrick E. Eddy et al., the entire disclosure of which is incorporated herein by reference. The term “built-in” vacuum system is intended to refer to vacuum systems that are plumbed into the building structure of a healthcare facility and is not intended to cover a vacuum pump mounted to a wall or other structure of the patient's room. “Healthcare facility” is intended to include hospitals, outpatient treatment facilities, doctors' offices, nursing homes, and any other facility in which healthcare services are provided.

The source of vacuum (whether it is an internal vacuum pump or a built-in central vacuum system) may have a vacuum between 0 and 600 mmHg. The vacuum from a built-in central vacuum system may be regulated by vacuum regulator 132 operating under control of suction control circuit 136 and may be selectively applied continuously or intermittently or may be interrupted by valve 134. The application of negative pressure to the wound site 250 can be actuated at predetermined time intervals or in response to wound site conditions such as an accumulation of fluid under the wound dressing 200. During an intermittent vacuum mode the apparatus may vent to atmosphere or supply low pressure oxygen to the wound during vacuum off time.

System 130 may further comprise third and fourth electrically conductive sensing elements 45a and 45b (FIGS. 4 and 5) disposed inside container 20 so as to be exposed to fluid collected in container 20, where third and fourth sensing elements 45a and 45b are located inside container 20 at a position corresponding to a nearly full fluid level at which point container 20 is nearly full of fluid, but not yet full of fluid. Control circuit 136 may be coupled to third and fourth sensing elements 45a and 45b for generating an initial alarm signal when control circuit 136 determines that current can flow between third and fourth sensing elements 45a and 45b as a result of fluid in container 20 reaching the nearly full fluid level and thereby completing a current path between third and fourth sensing elements 45a and 45b. An audible alarm generator 80a may be coupled to receive the initial alarm signal from control circuit 136 and to produce an audible alarm sound upon receiving the initial alarm signal. A visual alarm generator 90a may be coupled to receive the initial alarm signal from control circuit 136 and to produce a visible alarm upon receiving the initial alarm signal.

Although the application described herein of suction regulator/pump 131 is that of negative pressure wound treatment (NPWT), suction regulator 20 may be used in a variety of applications. The electronically controlled suction regulator is well-suited for use in healthcare facilities as a general safe method of filtering and regulating reduced pressure for procedures such as, but not limited to: Nasopharyngeal, tracheal, surgical, gastrointestinal, pleural, wound drainage, etc. The features that make suction regulator/pump 131 uniquely suited for NPWT is its ability to: (1) allow end user adjustment of the output suction characteristics (i.e., timing cycle, intermittent mode, continuous mode, pressure, etc.), (2) generate an alarm if fluid in a canister reaches a particular level, and (3) generate an alarm if the flow rate from the wound is too high (above a threshold level), which indicates a leak.

The electronically controlled suction regulator/pump 131 may be hung on a wall using preexisting brackets or may be placed on a bed using a clamp or pole, or be free standing with and without an optional base and an IV pole.

A filter/fluid trap that is permeable by gas only and not permeable by solids or liquids may be interposed between the vacuum source and container 20 to prevent solids or liquids from being introduced into the regulator/pump system, the conduits, or the vacuum source. The filter may be a porous polymer that impedes solids and liquids from passing but allows gaseous materials to pass. The filter may be a polymer or other natural substance. The filter may be single or plural, but may cover all conduits exiting container 20. An outlet conduit for fluid may be connected between an outlet port of the canister and the vacuum source, and the filter may be disposed in the container substantially at the interface between the outlet port and the outlet conduit.

A first pressure detector may be provided that is adapted to detect a pressure drop indicative of the filter being substantially covered/blocked by water or solids.

Suction regulator/pump 131 may further comprise an optional negative pressure detector disposed in the inlet conduit that may compare the measured pressure with a preset level to determine if the negative pressure/vacuum is at or above the preset pressure level. This system will work with a single conduit/tube and can aid in prevention of blockage without need for separate detection systems.

The suction regulator/pump electronics may be configured to time stamp the proximity switches position while identifying the operator. As explained further below, such time stamps and switch positions may be supplied to the healthcare facility's records database.

The electronics logic may be configured to protect patients by alarming if too much fluid is contained in the container in a pre-entered time frame.

According to one embodiment of the present invention, the system 130 is used for applying a negative pressure to a wound. This may be accomplished by connecting the outlet conduit of the suction regulator/pump 130 to the patient interface portion 200 (i.e., the portion to which a portable pump was previously attached) of the systems disclosed in U.S. Pat. Nos. 4,382,441, 4,392,858, 4,655,754, 4,826,494, 4,969,880, 5,100,396, 5,261,893, 5,527,293, 5,636,643, 5,645,081, 6,071,267, 6,117,111, 6,135,116, 6,142,982, 6,174,306, 6,345,623, 6,398,767, 6,520,982, 6,553,998, 6,814,079, 7,198,046, and 7,216,651, the entire disclosures of which are incorporated herein by reference.

FIGS. 9-13 relate to disposable wound dressings 200 that may be used in the inventive system. The disposable wound dressings shown in FIGS. 9-13 are described below and are described in commonly-assigned U.S. Provisional Patent Application No. 61/041,301, entitled “WOUND TREATMENT SYSTEM,” filed on Apr. 1, 2008, by Pat E. Eddy et al., the entire disclosure of which is incorporated herein by reference. FIGS. 9-12 show an example of one wound treatment system to which the various improvements may be implemented separately or in various combinations. FIG. 9 is a perspective view of a disposable wound dressing 200. Disposable wound dressing 200 includes wound drape 222 that includes an interior portion 224 surrounded by a perimeter 226. Drape 222 further includes a skin contact surface 228 with an adhesive coating 230. The drape may be made of membrane permeable, semi-permeable or non-permeable materials that are commercially available, an example being material referred to as TAGODERM®, which is available from the 3M (Minnesota Mining and Manufacturing) Company of St. Paul, Minn. A protective backing 223 is placed over the adhesive coating 230 on the skin contact surface 228 until drape 222 is ready for application.

Wound drape 222 may comprise a pair of panels 219 with inner, upturned edges 220 which can be adhesively joined together to form a seam 221, which extends transversely across drape 222 and projects generally upwardly therefrom. The panels 219 can be secured together at the seam 221 by the adhesive coating 230 to form the seam 221. Alternatively, drape 222 may be made of a single panel as described further below.

The vacuum conduit may include a tube or sheath 234 including a proximate end 36 located under drape 222 and a distal or free end 238. The tube 234 can be inserted through the seam 21 which forms an opening 232 between the panel edge strips 220 at approximately the center of the drape 222. If a single panel 219 is used such that no seam is present, a hole may be formed in the drape 222 for passage of the tube or for placement of an attachment pad or coupler (discussed below). A relatively short length of the tube 234 adjacent to its proximate end 236 is shown under the drape 222 in FIG. 10, but greater lengths of the tube 234 could be placed under the drape 222. As shown in FIG. 12, the tube proximate end 36 is open and adjacent to the proximate end 236 one or more openings are formed. The tube opening(s) 239 may project downwardly, i.e., away from the skin contact surface 228. The short length of the tube 234, which is located under drape 222, can be releasably secured to the skin contact surface 228 by the adhesive coating 230, preferably with the tube opening 239 facing downwardly. The tube 234 may have a length that is sufficient to extend to the vacuum source 242 or to the containment apparatus 241. Alternatively, a second tube may be attached to the free end 238 of the tube 234.

The tube 234 can comprise, for example, a flexible, plastic tube of the type that is commonly used as a percutaneous sheath for intravenous treatments. At its distal end 238, the tube 234 may be adapted for: (1) closure with a variety of suitable closure devices; (2) connection to various active and passive fluid collection devices for draining and evacuating fluid from the wound site; and (3) connection to various fluid source devices for actively and passively introducing fluid to the wound site.

FIG. 12 shows the tube distal end 238 fluidically communicating with a suction regulator 20 for actively draining fluid from the wound site.

The disposable wound dressing 200 may further include a wound dressing pad 225 between the wound site 250 and drape 222. The wound dressing pad 225 can comprise a variety of materials with varying properties such as: (1) absorbency; (2) wicking or capillary action; and (3) surface contact action. The wound dressing 225 is primarily located in a chamber 246 formed between the wound 250 and the drape 222.

In wound treatment systems such as the one described above, the wound dressing pad 225 is sized and shaped to fit in and over the wound to be treated, and thus the wound dressing is in direct contact with the wound. In prior systems, a gauze or foam is used as the wound dressing pad so as to allow air to flow around the wound. The air flow is caused by the application of a vacuum. Because the vacuum also tends to draw fluids from the wound and through the wound dressing pad, the wound tissue can grow into the wound dressing pad or otherwise stick to the wound dressing pad. This causes problems in that the wound does not heal properly and can also reopen when the wound dressing is removed or changed. In addition, the removal of a wound dressing pad that is stuck to the wound can be particularly uncomfortable for the patient.

Wound dressing pad 225 may be siliconized to allow tissue on and around the wound to form without growing into or onto the wound dressing pad or from otherwise sticking to the wound tissue. The wound dressing pad may include a natural fiber, polymer, foam (such as a granufoam-urethane base or whitefoam-PVA base), or other filler/support material. An example of a foam is a granufoam available from Kinetic Concepts, Inc. (KCI) of San Antonio, Tex. The filler/support material could be “siliconized.” This can occur by applying silicone to at least the surface of the filler/support material that directly contacts the wound, by impregnating the filler/support material with silicone, or by using a filler/support material that already integrally includes silicone or its equivalent. By using silicone or an equivalent, the wound can properly heal without the wound tissue growing into or sticking to the wound dressing pad. One commercially available material that may be used as the wound dressing is THERAGAUZE®, which is available from Soluble Solutions, LLC of Newport News, Va. The formulation of THERAGAUZE® is believed to be disclosed in U.S. Pat. No. 6,592,860, the entire disclosure of which is incorporated herein by reference. Alternatively, one may use foam that is seared to close cells on the foam surface adjacent the wound, or use a dual-density foam (two styles of foam together for different end effects). Specifically, the dual density foam pad 225 may include a larger cell foam layer and a smaller cell foam layer that contacts the wound. Foam pad 225 may further include an optional coating of a material such as silicone.

The silicone/seared foam may or may not be perforated or slit to allow vacuum, ambient or a positive pressure to pass through, and to allow liquids to pass. Whether to perforate or slit the silicone will depend upon the particular application and the nature of the filler/support material and how the silicone is provided.

The siliconized wound, seared, duel density dressing pads 225 may be coated with a medicated or non-medicated solution such as polypropylene, glycol and saline, silver, an anti-bacterial solution or the like, that may promote healing and/or reduce adhesion of tissue and fluids.

Alternatively, wound dressing pad 225 is made of a bio-absorbable material such that wound tissue growth into pad 225 becomes a positive condition rather than a negative condition as the pad may simply be left in place into the patient's body absorbs the pad.

The wound drape 222 may be any conventional drape material known to be used for vacuum-assisted wound treatment. The material may be a semi-permeable or impermeable flexible covering that may or may not have a valve/relief to the outside atmosphere. The wound drape may have one or more apertures for allowing a tube, attachment pad, or other coupler to be inserted for connection of the vacuum conduit and application of the vacuum to the wound. The application of the vacuum may be regulated and varied during a course of treatment. In addition, the vacuum may be intermittently applied.

The system may use a tube that has a plurality of apertures through its sidewalls at the end of the tube that extends into and under the wound drape. The end of the tube may lie between the drape and the wound dressing or it may extend into the wound dressing.

An attachment pad/coupler has been developed that includes a mechanical device to provide a visual acknowledgement of vacuum at a predetermined level at or near the wound site. Commonly assigned U.S. patent application Ser. No. 12/187,114, entitled “WOUND TREATMENT SYSTEM AND SUCTION REGULATOR FOR USE THEREWITH” and filed on Aug. 6, 2008 by Patrick E. Eddy et al. discloses various attachment pad/couplers that may be employed, the entire disclosure of which is incorporated herein by reference. An attachment pad such as those disclosed in U.S. Pat. Nos. 6,345,623, 6,553,998, and 6,814,079 may also be used with the inventive system. In addition, a TRACKPAD™ available from KCI may also be employed.

The above systems are disclosed as using a single container 20. When the container becomes full, the vacuum system is stopped until the healthcare professional overseeing the treatment of the patient can get to the room, remove and empty or dispose of the container, optionally return the container or install a new one, and restart the system. All of this takes time and interrupts the procedure. By using two containers, a first container can be used in the normal course, and when the full alarm signal is generated, a signal is sent to an electronically controlled valve that diverts the flow of fluid from the first container to the second container to thereby allow uninterrupted use. When the alarm is generated indicating the first container is full, the healthcare professional overseeing the treatment of the patient, can empty or replace the first container as was done previously, except that the system can keep operating with the fluid flowing to the second container. Upon returning the empty first container, the system can either automatically return the flow of fluid to the first container or continue the flow of fluid to the second container until such time that it becomes full—at which time the valve may be reactuated to divert the flow to first container.

Flow sensor 150 may be used to monitor the pressure of the vacuum and determines if a predetermined start up pressure lasts for a certain time. This feature (also known as “wound close technology”) allows one to monitor the progression of the wound to closure. This can be displayed on display screen 144 and would work as an initial start cycle function that can be done at a new wound site, change of dressing, or as a special cycle that will work when the wound site is at ambient/atmospheric pressure.

A valve mechanism may be provided at the attachment pad or elsewhere that allows ambient air to be vented to the wound at 1 or 2 psi whenever the vacuum is in an off interval of an intermittent cycle or the vacuum is removed.

The system may also be configured to a high flow (leak detection) alarm that is activated when the flow of air from the wound site is above a threshold.

Referring back to FIG. 7, optional transmitter/transceiver 172 may be provided to transmit information to a receiver/transceiver 176 that receives the information and provides it to an automated records database 174 of the healthcare facility. The information may include any one or more of the following: the times at which negative pressure was applied to the wound, the pressure applied, the intermittence cycles, the times at which the settings were changed along with the new settings, leak detection alarm times, full canister alarms times, and readings from flow sensor 150 which allows one to monitor the progression of the wound to closure. Transmitter/transceiver 172 may be coupled wirelessly or by wired connection such as USB. The database 174 may be a database such as a Cerner records database.

Transmitter/transceiver 172 may be coupled wirelessly or by wired connection to a nursing station alarm switchboard 178 so as to generate appropriate alarm signals at a nursing station that identifies the patient or room and the warning that a medical waste container is full or nearly full so that the staff can take appropriate action.

Each of the above-noted features may be implemented separately from the other features, or in combination with one or more of the other features.

The above description is considered that of the preferred embodiments only. Modification of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the Doctrine of Equivalents.

What is claimed is:

1. An apparatus for collecting medical waste fluids, comprising:
a medical waste collection container for collecting medical waste fluids;
first and second electrically conductive sensing elements disposed inside said container so as to be exposed to fluid collected in said container, where said first and second sensing elements are located inside said container at a position corresponding to a full fluid level at which point said container is full of fluid; and
an electronic circuit coupled to said sensing elements for generating a full alarm signal when said electronic circuit determines that current can flow between said first and second sensing elements as a result of fluid in said container reaching the full fluid level and thereby completing a current path between said first and second sensing elements.

2. The apparatus of claim 1 and further comprising an audible alarm coupled to receive the full alarm signal from said electronic circuit and to produce an audible alarm sound upon receiving the full alarm signal.

3. The apparatus of claim 2, wherein said audible alarm includes a piezo electric noise emitter.

4. The apparatus of claim 2 and further comprising a switch for silencing said audible alarm when actuated.

5. The apparatus of claim 2, wherein said audible alarm is mounted remotely from said container.

6. The apparatus of claim 1 and further comprising a visual alarm generator coupled to receive the full alarm signal from said electronic circuit and to produce a visible alarm upon receiving the full alarm signal.

7. The apparatus of claim 6, wherein said visual alarm generator includes an LCD visual indicator.

8. The apparatus of claim 6, wherein said visual alarm generator includes an LED visual indicator.

9. The apparatus of claim 6, wherein said visual alarm generator is mounted remotely from said container.

10. The apparatus of claim 1, wherein said sensing elements comprise copper leads.

11. The apparatus of claim 1 and further comprising a valve for interrupting a flow of medical waste fluid into said container in response to the full alarm signal.

12. The apparatus of claim 11 and further comprising third and fourth electrically conductive sensing elements disposed inside said container so as to be exposed to fluid collected in said container, where said third and fourth sensing elements are located inside said container at a position corresponding to a nearly full fluid level at which point said container is nearly full of fluid, but not yet full of fluid, wherein said electronic circuit is coupled to said third and fourth sensing elements for generating an initial alarm signal when said electronic circuit determines that current can flow between said third and fourth sensing elements as a result of fluid in said container reaching the nearly full fluid level and thereby completing a current path between said third and fourth sensing elements.

13. The apparatus of claim 12 and further comprising an audible alarm coupled to receive the initial alarm signal from said electronic circuit and to produce an audible alarm sound upon receiving the initial alarm signal.

14. The apparatus of claim 12 and further comprising a visual alarm generator coupled to receive the initial alarm signal from said electronic circuit and to produce a visible alarm upon receiving the initial alarm signal.

15. The apparatus of claim 1, wherein said electronic circuit is mounted within said container.

16. The apparatus of claim 1, wherein said electronic circuit is mounted remotely from said container.

17. The apparatus of claim 1, wherein said electronic circuit is powered by a battery.

18. The apparatus of claim 1 and further comprising a vacuum pump for drawing medical waste fluid into said container.

19. The apparatus of claim 1, wherein said container is made of a plastic that is generally frosted with the exception of a fluid level viewing window that is transparent.

20. A system for the treatment of wounds by applying a negative pressure to a wound site, comprising:
an electronically controlled suction regulator;
a valve connected to said suction regulator for supplying a suction at an output;
a medical waste collection container for collecting medical waste fluids and coupled to the output of said valve;
first and second electrically conductive sensing elements disposed inside said container so as to be exposed to fluid collected in said container, where said first and second sensing elements are located inside said container at a position corresponding to a full fluid level at which point said container is full of fluid; and
a control circuit coupled to said valve and said first and second sensing elements for generating control signals for controlling said valve and for generating a full alarm signal when said control circuit determines that current can flow between said first and second sensing elements as a result of fluid in said container reaching the full fluid level and thereby completing a current path between said first and second sensing elements, wherein said control circuit further generates a control signal to interrupt the supply of the suction at said output when a full alarm signal is generated.

21. The system of claim 20 and further comprising a vacuum pump coupled to said suction regulator.

22. The system of claim 20 and further comprising a wound dressing provided at the wound site and coupled to said valve, said wound dressing comprising:
a wound dressing pad for placing over the wound; and
a wound drape provided over said wound dressing pad and the wound site for securing said wound dressing pad and sealing the wound site for application of the negative pressure.

23. The system of claim 20 and further comprising third and fourth electrically conductive sensing elements disposed inside said container so as to be exposed to fluid collected in said container, where said third and fourth sensing elements are located inside said container at a position corresponding to a nearly full fluid level at which point said container is nearly full of fluid, but not yet full of fluid, wherein said control circuit is coupled to said third and fourth sensing elements for generating an initial alarm signal when said control circuit determines that current can flow between said third and fourth sensing elements as a result of fluid in said container reaching the nearly full fluid level and thereby completing a current path between said third and fourth sensing elements.

24. The apparatus of claim 23 and further comprising an audible alarm coupled to receive the initial alarm signal from said control circuit and to produce an audible alarm sound upon receiving the initial alarm signal.

25. The apparatus of claim 23 and further comprising a visual alarm generator coupled to receive the initial alarm signal from said control circuit and to produce a visible alarm upon receiving the initial alarm signal.

26. The system of claim 20, wherein said control circuit comprises an electronic circuit coupled to said first and second sensing elements, and a suction control circuit coupled to said electronic circuit and to said valve and said suction regulator.

27. The system of claim 20, wherein said electronic circuit is mounted within said container.

28. The system of claim 20, wherein said electronic circuit is mounted remotely from said container.

29. A method of detecting whether a container is full of medical waste fluids comprising the steps of:
providing first and second electrically conductive sensing elements inside the container so as to be exposed to fluid collected in the container, where the first and second sensing elements are located inside the container at a position corresponding to a full fluid level at which point the container is full of fluid; and
generating a full alarm signal when electrical current can flow between the first and second sensing elements as a result of fluid in the container reaching the full fluid level and thereby completing a current path between the first and second sensing elements.
